(19) United States
(12) Patent Application Publication
(10) Pub. No.: US 2022/0387645 A1
(43) Pub. Date: Dec. 8, 2022

(54) METHODS OF TREATMENT OF VOLATILE ORGANIC COMPOUNDS USING CHLORINE DIOXIDE

(71) Applicant: PROKURE SOLUTIONS, LLC, Phoenix, AZ (US)

(72) Inventors: Alex Joseph Cushman, Phoenix, AZ (US); Bret Bernard Lorenz, Phoenix, AZ (US)

(21) Appl. No.: 17/776,198

(22) PCT Filed: Nov. 12, 2020
(86) PCT No.: PCT/US20/60273

§ 371 Date: May 11, 2022

Related U.S. Application Data
(60) Provisional application No. 62/933,860, filed on Nov. 11, 2019.

(51) Int. Cl. A61L 2/20; D06M 11/30; D06M 23/06; A61L 2/18

(52) U.S. Cl. CPC A61L 2/20; D06M 11/30; D06M 23/06; A61L 2/18; A61L 2101/06

(57) ABSTRACT

In various embodiments, methods of treating a space to reduce a concentration of volatile organic compounds present in the space using chlorine dioxide are provided. A method can include application of aqueous and gaseous chlorine dioxide solutions within the space or to materials located within the space. Treatment of materials that emit volatile organic compounds with chlorine dioxide can reduce the emission rate or shorten the volatile organic compound emission cycle of the material. Soft surface substrates such as carpeting materials can be treated with chlorine dioxide to reduce volatile organic compound emission and/or to reduce the number of microorganisms present in the material.

### CROSS REFERENCE TO RELATED APPLICATIONS

This application is a National Stage Application of PCT/US20/60273 filed on Nov. 12, 2020, which claims priority to and the benefit of U.S. Ser. No. 62/933,860 filed on Nov. 11, 2019 and entitled Methods of Treatment of Volatile Organic Compounds Using Chlorine Dioxide, which applications are incorporated herein in their entirety for any purpose,

### FIELD

The present disclosure relates to methods for treating an area to reduce the concentration of volatile organic compounds.

### BACKGROUND

Indoor air pollution has been identified as one of five major environmental pollution factors that pose a significant risk to human health, with particular risks in developing countries. See Lv et al., 2016, Experimental and simulation study on bake-out with dilution ventilation technology for building materials, J. Air Waste Man. Assoc., 66(11): 1098-1108 and references therein, all of which are hereby incorporated by reference for any purpose.

Volatile organic compounds (VOCs) include petroleum-based chemicals which are often found at significant, levels in residential and commercial buildings as a result of VOC emission from building materials, furnishings, and other articles and agents that may be introduced to an enclosed space in a residential or commercial building. Various consumer and commercial products undergo a process of emission of VOCs commonly referred to as outgassing or off-gassing. For example, volatile organic compounds can be produced from synthetic fragrances (as found in soaps, candles, air fresheners, incense and potpourri), paint, carpet, furnishings, glues, plastics, pressed wood products (such as plywood and particle board) and other consumer products, construction, and decorating materials.

One example of a VOC is formaldehyde. Formaldehyde is found in many building materials such as caulks and adhesives, paint, furniture, etc. Formaldehyde is a desensitizing substance that lowers the ability to recognize or sense other potentially harmful chemicals. Prolonged exposure to formaldehyde can result in a person experiencing symptoms such as headaches, numbness or tingling of extremities, lightheadedness, inability to concentrate, anxiety, and depression. Other common VOCs include benzene, toluene, xylene, and related compounds.

VOCs can include very volatile and semi-volatile organic compounds (VVOCs and SVOCs, respectively). Newly constructed and renovated buildings may be primarily affected by emission of intermediate VOCs for a period of several days to weeks following construction or renovation. Total VOC (TVOC) emission rates per unit volume of enclosed space decay over a time period following construction or renovation and stabilize to levels influenced predominately by SVOCs and VOCs introduced by occupancy and use of the space. See Holos et al. 2019, VOC emission rates in newly built and renovated buildings, and the influence of ventilation—a review and meta-analysis, Int. J. of Ventilation, 18(3): 153-166 and references therein, all of which are hereby incorporated by reference for any purpose.

VOC emission and accumulation in a space can be reduced by methods such as ventilation; however, where a source of formaldehyde or other volatile organic compound is organic matter such as mold, off-gassing can be continuous and persistent. Volatile organic compounds that are off-gassed as waste products of mold can be more dangerous to an individual's health than mold spores contained in the air.

In addition to producing deleterious physical side effects for human occupants. the VOCs can also produce unpleasant or noxious odors. Odors are often associated with levels of VOCs within a space. A reduction in VOC levels in such spaces will result in a corresponding reduction in odors.

Existing methods for removing volatile organic compounds rely on systems and apparatus that require set up and installation time, manpower and associated hours to perform the removal methods, periods in which a space may not be occupied, and associated expenses.

The use of fumigants for controlling, killing or preventing microbiological contamination (e.g., bacteria, fungi, viruses, mold spores, algae and protozoa); retarding, preventing, or controlling biochemical decomposition; controlling respiration, deodorizing and/or retarding and preventing chemotaxis, is known. Such fumigants include, but are not limited to, chlorine dioxide, sulfur dioxide, nitrogen dioxide, nitric oxide, nitrous oxide, carbon dioxide, hydrogen sulfide, hydrocyanic acid, and dichlorine monoxide. Chlorine dioxide (CD) has been used as a fumigant but there are concerns with its use as a gas phase sterilant at high concentrations. Concentrated chlorine dioxide is highly oxidizing and can cause corrosion or oxidative damage to materials located within an enclosed structure upon completion of a fumigation treatment.

New methods for effectively and efficiently reducing the concentration of VOCs in enclosed residential and commercial spaces and for reducing VOC emission rates from materials used in construction and renovation such as floor coverings without causing damage or further side effects are desirable.

### SUMMARY

In aspects of the disclosure, a method of treating a space to reduce the concentration of volatile organic compounds is provided. In aspects, an aqueous or gaseous solution comprising chlorine dioxide is applied to the space in which volatile organic compounds are emitted and the solution is applied or dispersed in a manner effective to reduce the rate of emission or concentration of volatile organic compounds in the space. In various embodiments, a method of treating a space to reduce volatile organic compound emissions can comprise treating a surface located inside the space.

In aspects of the disclosure, methods of treating a space to reduce a concentration of a volatile organic compound (VOC) are provided. In embodiments, a method may include determining a first concentration of a first VOC at a first time and applying a chlorine dioxide treatment in the space for a treatment period in response to the first concentration of the first VOC. In embodiments, the chlorine dioxide treatment is effective to reduce the first concentration of the first VOC to a second concentration at a second time that is lower than the first concentration as compared to a non-treated space. In embodiments, the second concentration is one of about 10%, or about 15%, or about 20%, or about 25%, or about 30%, or about 35%, or about 40%, or about 45%, or about 50%, or about 55%, or about 60%, or about 65%, or about 70%, or about 75%, or about 80%, or about 85%, or about 90%, or about 95%, or about 100% lower than the first concentration. In embodiments, the method further includes determining a first concentration of a second VOC at a first time. In embodiments, the chlorine dioxide treatment is effective to reduce the first concentration of the second VOC to a second concentration at the second time that is lower than the first concentration. In embodiments, the space comprises one of a residential and a commercial building interior space. In embodiments, the space comprises a carpeted surface. In embodiments, the applying a chlorine dioxide step comprises one of applying an aqueous chlorine dioxide solution to the carpeted surface and dispersing a gaseous chlorine dioxide solution in the space. In embodiments, the space comprises a floor covering, where the first VOC is emitted from the floor covering, and where the applying a chlorine dioxide step comprises applying a gaseous chlorine dioxide treatment. In embodiments, the method is performed subsequent to installation of the floor covering in the space. In embodiments, the first VOC is emitted from one of a floor covering material, an underlayment material, and a floor covering adhesive. In embodiments, the first VOC is emitted from one of a floor covering material, an underlayment material, and a floor covering adhesive. In embodiments, the first VOC is emitted from one of an underlayment material and a floor covering adhesive, and wherein the one of the underlayment material and the floor covering adhesive is positioned beneath a floor covering material.

In aspects of the disclosure, methods of reducing a volatile organic compound (VOC) emission rate of a material are provided. In embodiments, a method may include determining a first VOC emission rate of a material at a first time; applying a chlorine dioxide treatment to a material to produce a treated material; and determining a second VOC emission rate of the treated material at a second time. In embodiments, the chlorine dioxide treatment produces a reduced VOC emission rate of the treated material where the second VOC emission rate of the treated material is lower than the first VOC emission rate of the material. In embodiments, a method further includes, reserving an untreated portion of the material; determining an untreated material VOC emission rate of the untreated portion of the material at the second time; and comparing the second VOC emission rate of the treated material to the untreated material VOC emission rate to determine a VOC emission rate reduction effect of the chlorine dioxide treatment. In embodiments, the material comprises a carpeting material. In embodiments, the chlorine dioxide treatment is suitable to achieve the reduced VOC emission rate without a substantial effect on one of a material color, a material physical integrity, a material physical performance (stain resistance, wear resistance, color fade resistance), and a material physical attribute (strength, softness, flexibility, resiliency). In embodiments, applying a chlorine dioxide treatment is performed at one of a pre-tufting stage (undyed or dyed yarn), a post-tufting and dying stage, a post-drying and pre-finishing stage, a post-finishing and pre-packaging stage, a packaging stage, and a post-installation stage. In embodiments, the first time is less than one of about 4 hrs, about 8 hrs, about 12 hrs, about 16 hrs, about 20 hrs, and about 24 hrs after installation of the carpeting material in a space. In embodiments, the second time is less than one of about 24 hrs, about 36 hrs, about 48 hrs, about 60 hrs, about 72 hrs, and about 96 hrs.

In aspects of the disclosure, methods for treating a substrate with a chlorine dioxide treatment, where the chlorine dioxide treatment is effective to reduce a TVOC emission rate of the substrate are provided. In embodiments, the chlorine dioxide treatment does not produce a detrimental impact on the substrate are provided.

In aspects of the disclosure, methods comprising treating a substrate with a chlorine dioxide treatment in situ in one of a space for a treatment period are provided. In embodiments, the space may have one of a residential space or a commercial space. In embodiments, the chlorine dioxide treatment is compatible with human occupancy of the space during the treatment period. In embodiments, the chlorine dioxide treatment is effective to reduce an emitted TVOC quantity in the space as compared to an untreated substrate in an equivalent space. In embodiments, the chlorine dioxide treatment does not produce a detrimental impact on the substrate.

In aspects of the disclosure, methods for treating a soft surface substrate comprising a first microorganism located in the soft surface substrate with a chlorine dioxide treatment for a treatment period are provided. In embodiments, the first microorganism is viable prior to the chlorine dioxide treatment. In embodiments, the chlorine dioxide treatment is effective to render the first microorganism non-viable. In embodiments, the chlorine dioxide treatment does not produce a detrimental impact on the soft surface substrate. In embodiments, the chlorine dioxide treatment comprises application of gaseous chlorine dioxide in one of a residential and a commercial interior space enclosing the soft substrate.

In aspects of the disclosure, methods for reducing a number of microorganisms in a material are provided. In embodiments, a method includes determining a first number of microorganisms in a material at a first time, applying a chlorine dioxide treatment to a material to produce a treated material, and determining a second number of microorganisms in the treated material at a second time. In embodiments, the chlorine dioxide treatment produces a reduced number of microorganisms in the treated material wherein the second number of microorganisms in the treated material is lower than the first number of microorganisms in the material. In embodiments, the number of microorganisms is determined using a quantitative assay.

In aspects of the disclosure, methods for reducing a quantity of a microorganism metabolite in one of a material or a space are provided. In embodiments, a method may include determining a first quantity of a microorganism metabolite in one of a material or a space at a first time, applying a chlorine dioxide treatment to a material to produce one of a treated material and a treated space, and determining a second quantity of the microorganism metabolite in one of the treated material and the treated space at a second time. In embodiments, the chlorine dioxide treatment is effective to reduce the quantity of the microorganism metabolite in the one of the treated material and the treated space and wherein the second quantity of the microorganism metabolite is lower than the first quantity of the microorganism metabolite. In embodiments, the microorganism metabolite comprises a microbial VOC.

### DETAILED DESCRIPTION

In aspects of the disclosure, methods of treating a material or an area to reduce the concentration or rate of emission of volatile organic compounds (VOCs) are presented.

In aspects of the disclosure, VOCs can be emitted from a material during a process known as off-gassing. During the process of off-Passing, volatile organic compounds are emitted from a material or product over time. Off-gassing may occur in a wide array of products and materials, including solid and liquid materials. Off-gassing may result in health issues and symptoms for human occupants of a space in which VOCs are emitted. In some cases, exposure to VOCs produced during the off-gassing process may cause short term health issues including, but not limited to, headaches, dizziness, fatigue, nausea, eye, nose and throat irritation, problems with vision and memory problems. In some instances, off-gassing may cause longer term health issues including, but not limited to, cancer, respiratory problems (for example asthma), heart disease, kidney damage, liver damage, neurological disorders, reproductive disorders, developmental disorders (for example, in young children or babies exposed to VOCs), endocrine disorders and central nervous system damage.

In aspects of the disclosure, VOCs include, but are not limited to, formaldehyde, chloroform, phthalates, acetone, ozone, ethanol, chemical flame retardant, methylene chloride, benzene, isocyanates, styrene, and perchloroethylene (for example, in dry cleaning solutions). Any VOC or other volatile chemical compounds (for example, volatile sulfur compounds) now known or identified in the future are within the scope of the present disclosure.

In aspects of the disclosure, VOCs may be emitted from paints and painting supplies, floor and wall covering materials (including, for example, carpet, laminate, underlayment, tile, wallpaper, adhesives), furniture, cleaning supplies, refinishing and decorating supplies (for example, glues, adhesives, paint strippers, varnishes), building materials, pesticides, aerosol sprays, cosmetics, copiers and printers, burning wood, coal or natural gas, and smoking (for example, cigarettes, vapes etc. and the like).

As a non-limiting example, carpeting can release chemicals from the backing used on carpets during the off-gassing process. Carpet backing can be made from polyvinyl chloride (PVC), polyurethane or latex. The adhesive used to affix carpets to the floor also contains VOCs, some of which have been linked to leukemia and lymphoma. The adhesive can include plasticizers, fillers, thickeners, surfactants, hardeners, and many other ingredients. Some carpet adhesives are wet-applied epoxy or acrylic systems. Most epoxy adhesive resins are formed using a combination of bisphenol A and epichlorohydrin, both of which have been identified as toxic. Acrylic adhesives can include nonylphenol ethoxylate (NPE) surfactants which break down into toxic nonylphenols. Carpeting can emit VOCs during the off-gassing process for five years or longer with the highest concentration emitted during the first several months after installation.

In various aspects of the present disclosure, chlorine dioxide (CD) may be used to reduce VOC emissions from a material or to reduce VOC concentrations in a heated space. Without wishing to be bound by theory, inventors believe that application of CD in liquid or gaseous form to materials known to emit VOCs may accelerate degradation of VOCs on or within the source material prior to emission of VOCs from the material or contemporaneously with emission of VOCs from the source material. For example, application of CD to a carpet material may be suitable to reduce the emission of VOCs from the carpet material and/or to accelerate a VOC emission decay rate of the carpet material. Gaseous CD may be particularly effective in penetrating deep into carpeting material and degrading VOC compounds within the material due to the very small size of the CD molecule. Likewise, gaseous CD may be effective in reducing VOC emissions of upholstered furniture, mattresses, and other objects comprised of porous materials and fabrics. Moreover, application of an aqueous CD solution, for example as a spray or aerosol, may be effective via both contact by the solution as well as by gaseous CD molecules released from evaporating CD solution.

As used herein specifically in the context of application of an aqueous CD solution to carpet material, application of an aqueous CD solution can mean spraying or otherwise, applying the solution to the carpet with brushing or working to obtain deep fiber penetration. The solution is preferably contacted uniformly with the carpet in an amount sufficient to wet a substantial portion of the carpet fibers. Spraying can be carried out using, for example, a distribution wand or a power sprayer. One or more surfactants may be added to the cleaning solution to aid in wetting the carpet fibers.

The amount of a CD solution applied to a carpet may vary with the fiber composition, depth of pile and other factors related to carpet construction. In various embodiments, an amount may be sufficient to substantially wet the carpet fibers without forming a pool of liquid on the carpet substrate. Application rates of about 1 gallon per 50-200 sq. ft. (4.6-18.6 sq. meters) may be effective for a large range of carpet materials.

A CD treatment may be tested on a carpet material to be treated to ensure there is no substantial or detectable physical damage to the carpet material, including a detrimental impact on, for example, one of the carpet material's strength, softness, flexibility, resiliency, stain resistance, wear resistance, and color fade resistance.

A CD treatment may be tested on a carpet material to be treated to ensure there is no substantial or detectable damage to the carpet color. A wide variety of dyes and dye classes have been used on carpets, and some may he sensitive to cleaning agents, particularly when they are used at higher concentrations.

In aspects of the disclosure, treatment with chlorine dioxide may reduce VOC emission rate without a substantial effect on one of a material color, a material physical integrity, a material physical performance and a material physical attribute. In embodiments, a material physical performance may include, but not be limited to, stain resistance, wear resistance, and color fade resistance. In embodiments, a material physical attribute may include, but not be limited to, strength, softness, flexibility, and resiliency.

In various embodiments, a CD solution may be brushed or worked into the carpet to obtain deep fiber penetration. This can be effectively done using a carpet scrubbing machine with mechanically driven brushes or by hand brushing.

In various embodiments, a CD solution may be applied and allowed to remain on a treated carpet material for a defined treatment period. A CD solution may be removed after the defined treatment period. In various embodiments, a treatment may be suitable to effectively reduce VOC emissions from the treated carpet material without a negative impact on the treated carpet material.

In various embodiments, a CD solution may be delivered to a space to be treated by dispersing an aerosol of a CD solution to the space. An aerosolized CD solution can comprise a gaseous suspension of small liquid droplets of a CD solution.

Aerosols can be generated by a number of devices, including cold foggers, thermal foggers, impeller foggers, ultrasonic foggers, pressurized canisters, ultrasonic foggers, nebulizers (e.g., jet nebulizers, ultrasonic nebulizers, vibrating disc nebulizers), and other devices known in the art. Any suitable device may be used to disperse a CD solution in aerosol form in accordance with various embodiments of the present disclosure.

In aspects of the disclosure, the methods of the disclosure may be used to treat buildings including, but not limited to, new and existing construction, buildings undergoing renovation, group housing (for example, apartments, condos, dorm rooms, assisted living facilities, hotels, hostels), fitness facilities, and office spaces.

In aspects of the disclosure, the methods of treatment with CD may be effective to reduce the levels of VOCs to a level that is regarded as acceptable under the environmental protection agency (EPA) programs and certifications. In some aspects, the VOC levels are reduced allowing a product to be certified under certification programs now existing or implemented in the future that include, but are not limited to, the GreenLabel Plus (GLP), the California Gold Sustainable Carpet Standard, NSF/ANSI 140, Cradle-to Cradle (Silver-level), Oeko-Tex 100, the International Living Future Institutes Living Building Challenge (LBC) Red List, Germany's Blue Angel Label, Double Green and other applicable certification programs. The removal or reduction of VOCs in certain products may increase an ability to recycle and reuse the product. For example, carpeting having lower levels of VOCs can be recycled and reused which has an environmental impact in that the carpet does not end up in an incinerator or landfill.

In aspects of the disclosure, the methods disclosed may reduce a rate of emission of VOCs from a treated material. In various embodiments, a method of treatment of a material with CD can reduce a VOC emission rate by at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or about 100%.

In some aspects, treatment of a space containing a VOC at a VOC concentration with CD can reduce the concentration of the VOC to a level that is below detection using conventional detection methods. In aspects, the concentration of a VOC can be reduced to a level that is below the threshold level allowed under the Immediately Dangerous to Life or Health (IDLH) concentration or the Threshold Limit Value (TLV).

In aspects of the disclosure, the methods of reducing an emission rate of a VOC can be applied to a material or product prior to use or installation of the material or product in a space. In other aspects, the methods of reducing an emission rate of a VOC can be applied to a material or product after installation of the material or product in a space.

In various aspects, a product or material, or a space, or both may be treated with CD to reduce a VOC emission rate of a product or material or to reduce a concentration of a VOC or a TVOC concentration in a space.

In various aspects, treatment of a material can comprise application of CD directly to the material, such as by spraying an aqueous CD solution on a material, or treatment of a material can comprise indirect treatment of the material, such as by generation of CD gas in a space or environment in which a material is located, thereby causing exposure of the material in the treated space to gaseous CD.

In aspects of the disclosure, the concentration of VOCs may be measured using conventional methods known to determine the concentration of VOCs present after treatment using the methods of the disclosure. In aspects of the disclosure, the efficacy of the removal of VOCs using the methods of the disclosure may be tested using process challenge agents introduced during treatment as a proxy to determine treatment efficacy. In some aspects, a component that has an odor may be placed onto the surface area to be treated and measured prior to and after treatment to determine the rate of odor elimination using the methods of the disclosure. For example, hydrogen sulfide, β-mercaptoethanol, or some similar thiol or non-thiol odorant or process challenge agent may be used as proxies to determine the efficacy of removal of such components.

In aspects of the disclosure, the CD treatment methods of the disclosure may be effective to reduce the exposure of an individual human occupant of a space to a VOC when the space or a material in the space is treated in accordance with method disclosed herein. In aspects, the methods of the disclosure can reduce an individual's exposure to a VOC by at least about 10% to 100% compared to an untreated material or space. In aspects, an individual's exposure to VOCs is reduced by at least about 10%, 20%, 30%, 40%, 50%, 60%, 70%, 80%, 90%, 95%, 96%, 97%, 98%, 99%, or about 100%. In various embodiments, a CD treatment method may be performed during routine or continuous human use or occupancy, and such CD treatment may be permissible under applicable regulatory standards.

In aspects of the disclosure, the methods of the disclosure may be effective to reduce a VOC emission rate from a treated material by at least about 10%, 20%, 30%, 40%, 50%, 60%, 70%, 80%, 90%, 95%, 96%, 97%, 98%, 99%, or about 100% per unit treated material surface area per unit time as compared to an untreated material.

In aspects of the disclosure, the methods disclosed do not require an increase in the humidity in the space to be treated.

In aspects, the presence of chlorine dioxide gas is at a concentration that does not pose harm or potential harm to an individual. In aspects, the chlorine dioxide concentration is below a safe human occupancy threshold. In aspects, the chlorine dioxide concentration is below a threshold compatible with extended or continuous human exposure. In aspects, the treatment with chlorine dioxide gas does not require a period of time after treatment before an individual can re-enter or return to the treated space. In some aspects, the methods of the disclosure provide a treatment method that reduces the amount of time required between treatment of the space and reduction in the presence of VOCs such that an individual may return to the area after treatment In some aspects, the time between treatment and an individual returning to the treated space is less than about two hours. In aspects, the time between treatment and an individual returning to the treated space (the “Restricted Entry Interval” or “REI”) is less than about 2 hours, 1.5 hours, 1 hour, 45 minutes, 30 minutes, 15 minutes, or about 5 minutes. In certain aspects, the REI is less than about 30 minutes, 25 minutes, 20 minutes, 15 minutes, 10 minutes, 5 minutes, or about 1 minute.

In aspects of the disclosure, the application of a chlorine dioxide treatment may be performed at one of a pre-tufting stage (for example, undyed or dyed yarn), a post-tufting and dying stage, a post-drying and pre-finishing stage, a post-finishing and pre-packaging stage, a packaging stage, and a post-installation stage.

In aspects of the disclosure involving treatment of a carpeting material following installation in a space, a first time may be less than about 4 hours (hrs), about 8 hrs, about 12 hrs, about 16 hrs, about 20 hrs and about 24 hrs after installation of carpeting material in a space.

In aspects of the disclosure involving treatment of a carpeting material following installation in a space, a second time may be less than about 24 hrs, about 36 hrs, about 48 hrs, about 60 hrs, about 72 hrs, and about 96 hrs.

In aspects of the present disclosure, treatment of an enclosed environment or a surface covering (pre-installation or post-installation) with chlorine dioxide does not result in physical damage to a treated material or materials in a treated environment.

In aspects of the present disclosure, treatment of an enclosed environment or a surface covering (pre-installation or post-installation) with chlorine dioxide can produce a shortened VOC emission cycle in the enclosed environment. In aspects, a shortened VOC emission cycle can be achieved by treatment with chlorine dioxide in the absence of a bake-out process, ventilation process, dilution process, photocatalysis, activated carbon adsorption, or similar methods that may be used to remove or decrease VOCs in an enclosed environment. In various embodiments, shortening a VOC emission cycle can be achieved using a method comprising a combination of chlorine dioxide treatment and other methods such as bake out and/or ventilation processes.

In aspects of the disclosure, method of treatment may also result in sanitization or disinfection of a treated material by reducing a number of viable microorganisms in or on the treated material. In various embodiments, a treated material can comprise a soft surface or porous material, such as carpeting, upholstery, mattresses, draperies, artificial turf, and the like. Soft surface or porous material goods can comprise microorganisms located within the soft surface or porous material, and such microorganisms may be difficult to effectively remove or disinfect with standard cleaning procedures, while more robust chemical and/or physical measures may detrimentally impact the treated material or article and/or may be expensive, labor intensive, or create separate health hazards or inconveniences. In various embodiments, treatment with gaseous or liquid CD may be effective to reduce a number of viable microorganisms in or on a soft surface or porous material with substantially no negative effect on the treated material.

In various aspects of the disclosure, carpet material may provide conditions conducive to microbial growth and dust mites, for example, the presence of increased moisture content in a carpet material may provide a hospitable environment for microbial growth and dust mites. In embodiments, the presence of microorganisms may provide a source for chemical contaminants, such as microbial metabolites or toxins or may increase the amount of non-volatile, semi-volatile or volatile organic compounds present in an environment. The presence of a microorganism in a material may be detected using any suitable detection method, including various conventional detection methods disclosed herein. In embodiments, a quantitative assay may be used to determine the number of microorganisms in a material. In embodiments, quantitative assays may include, but not be limited to, cell counts, infectivity titers, microscopy, spectroscopy (for example, infrared spectroscopy, Raman spectroscopy), endpoint dilution assays, hemagglutination assays, flow cytometric assays, cell viability assays for example, immunofluorescence, polymerase chain reaction (PCR), reverse transcription-quantitative polymerase chain reaction (RT-qPCR), digital polymerase chain reaction (dPCR), plaque assays, enzyme-linked immunosorbent assays (ELISA), and the like. In embodiments, microorganisms and/or microbial metabolite chemical contaminants may be removed or reduced by treatment with gaseous or liquid CD. In embodiments, the effectiveness of decontamination of microorganisms in a material treated with CD may be measured as a probability of sterility for the material treated. This probability is the sterility assurance level (SAL) of the material and is defined as the probability of a single viable microorganism occurring on a material after sterilization. SAL is normally expressed as 10-n.

In various embodiments, a microorganism that may be present in a material includes, but is not limited to, bacteria, fungi, viruses, algae and protista. Microorganisms can produce metabolites that are known to be toxic or irritant to human occupants of a space. For example, mycotoxins, non-volatile secondary fungal metabolites capable of causing negative health effects, and other fungal secondary metabolites may be present in a carpet material. Mycotoxins include, but are not limited to, products of fungi such as aflatoxins and ochratoxins. Various bacteria produce microbial VOC (MVOC) metabolites that are known environmental irritants. In various embodiments, the presence of microorganisms in a material or a space and/or metabolites and other organic compounds produced by microorganisms may be reduced using various methods described herein.

In various embodiments, environmental allergens may be present in a material. Environmental allergens may include, but not be limited to, pollen, mold, dust, dust mites, pet dander, and arthropods. In embodiments, the concentration of environmental allergens in an environment may be reduced by treating the environment or a material in the environment with gaseous or liquid CD. In embodiments, the concentration of environmental allergens may be reduced to low or non-detectable levels that may reduce negative health effects that may be caused by such allergens, for example, asthma and other respiratory conditions.

### Embodiments

In an embodiment, a method of treating a space to reduce a concentration of a volatile organic compound (VOC) comprising: determining a first concentration of a first VOC at a first time; and applying a chlorine dioxide treatment in the space for a treatment period in response to the first concentration of the first VOC; wherein the chlorine dioxide treatment is effective to reduce the first concentration of the first VOC to a second concentration at a second time that is lower than the first concentration as compared to a non-treated space.

In an embodiment, the second concentration is one of about 10%, or about 15%, or about 20%, or about 25%, or about 30%, or about 35%, or about 40%, or about 45%, or about 50%, or about 55%, or about 60%, or about 65%, or about 70%, or about 75%, or about 80%, or about 85%, or about 90%, or about 95%, or about 100% lower than the first concentration.

In an embodiment, the method further comprises determining a first concentration of a second VOC at a first time, and wherein the chlorine dioxide treatment is effective to reduce the first concentration of the second VOC to a second concentration at the second time that is lower than the first concentration.

In an embodiment, the space comprises one of a residential and a commercial building interior space.

In an embodiment, the space comprises a carpeted surface, and wherein the applying a chlorine dioxide step comprises one of applying an aqueous chlorine dioxide solution to the carpeted surface and dispersing a gaseous chlorine dioxide solution in the space.

In an embodiment, the space comprises a floor covering, wherein the first VOC is emitted from the floor covering, and wherein the applying a chlorine dioxide step comprises applying a gaseous chlorine dioxide treatment.

In an embodiment, the method is performed subsequent to installation of the floor covering in the space.

In an embodiment, the first VOC is emitted from one of a floor covering material, an underlayment material, and a floor covering adhesive.

In an embodiment, the first VOC is emitted from one of an underlayment material and a floor covering adhesive, and wherein the one of the underlayment material and the floor covering adhesive is positioned beneath a floor covering material.

In an embodiment, a method of reducing a volatile organic compound (VOC) emission rate of a material comprising: determining a first VOC emission rate of a material at a first time; applying a chlorine dioxide treatment to a material to produce a treated material; and determining a second VOC emission rate of the treated material at a second time; wherein the chlorine dioxide treatment produces a reduced VOC emission rate of the treated material wherein the second VOC emission rate of the treated material is lower than the first VOC emission rate of the material.

In an embodiment, the method further comprises: reserving an untreated portion of the material; determining an untreated material VOC emission rate of the untreated portion of the material at the second time; and comparing the second VOC emission rate of the treated material to the untreated material VOC emission rate to determine a VOC emission rate reduction effect of the chlorine dioxide treatment.

In an embodiment, the material comprises a carpeting material.

In an embodiment, the chlorine dioxide treatment is suitable to achieve the reduced VOC emission rate without a substantial effect on one of a material color, a material physical integrity, a material physical performance (stain resistance, wear resistance, color fade resistance), and a material physical attribute (strength, softness, flexibility, resiliency).

In an embodiment, the applying a chlorine dioxide treatment is performed at one of a pre-tufting stage (undyed or dyed yarn), a post-tufting and dying stage, a post-drying and pre-finishing stage, a post-finishing and pre-packaging stage, a packaging stage, and a post-installation stage.

In an embodiment, wherein the first time is less than one of about 4 hrs, about 8 hrs, about 12 hrs, about 16 hrs, about 20 hrs, and about 24 hrs after installation of the carpeting material in a space.

In an embodiment, the second time is less than one of about 24 hrs, about 36 hrs, about 48 hrs, about 60 hrs, about 72 hrs, and about 96 hrs.

In an embodiment, a method comprising treating a substrate with a chlorine dioxide treatment, wherein, the chlorine dioxide treatment is effective to reduce a TVOC emission rate of the substrate, and wherein the chlorine dioxide treatment does not produce a detrimental impact on the substrate.

In an embodiment, a method comprising: treating a substrate with a chlorine dioxide treatment in situ in one of a space for a treatment period; wherein the space comprises one of a residential space or a commercial space; wherein the chlorine dioxide treatment is compatible with human occupancy of the space during the treatment period, wherein the chlorine dioxide treatment is effective to reduce an emitted TVOC quantity in the space as compared to an untreated substrate in an equivalent space, and wherein the chlorine dioxide treatment does not produce a detrimental impact on the substrate.

In an embodiment, a method comprising: treating a soft surface substrate comprising a first microorganism located in the soft surface substrate with a chlorine dioxide treatment for a treatment period; wherein the first microorganism is viable prior to the chlorine dioxide treatment, wherein the chlorine dioxide treatment is effective to render the first microorganism non-viable, and wherein the chlorine dioxide treatment does not produce a detrimental impact on the soft surface substrate.

In an embodiment, the chlorine dioxide treatment comprises application of gaseous chlorine dioxide in one of a residential and a commercial interior space enclosing the soft substrate.

In an embodiment, a method for reducing a number of microorganisms in a material comprising: determining a first number of microorganisms in a material at a first time; applying a chlorine dioxide treatment to a material to produce a treated material; and determining a second number of microorganisms in the treated material at a second time; wherein the chlorine dioxide treatment produces a reduced number of microorganisms in the treated material wherein the second number of microorganisms in the treated material is lower than the first number of microorganisms in the material.

In an embodiment, the number of microorganisms is determined using a quantitative assay.

In an embodiment, a method for reducing a quantity of a microorganism metabolite in one of a material or a space comprising: determining a first quantity of a microorganism metabolite in one of a material or a space at a first time; applying a chlorine dioxide treatment to a material to produce one of a treated material and a treated space; and determining a second quantity of the microorganism metabolite in one of the treated material and the treated space at a second time; wherein the chlorine dioxide treatment is effective to reduce the quantity of the microorganism metabolite in the one of the treated material and the treated space and wherein the second quantity of the microorganism metabolite is lower than the first quantity of the microorganism metabolite.

In an embodiment, the microorganism metabolite comprises a microbial VOC.

### EXAMPLES

### Example 1

### Determination of Effect of CD on VOCs

Various common industrial VOCs are released into sealed chambers at known concentrations or at set emission rates corresponding to concentrations or emission rates observed in newly constructed or newly renovated buildings. The VOC concentrations in the atmosphere of the sealed chambers are measured at regular time intervals. Chamber-based environmental condition simulation and testing is performed using standard conventional methods such as ASTM D5116-17, Standard Guide for Small-Scale Environmental Chamber Determinations of Organic Emissions from Indoor Materials/Products (ASTM International, West Conshohocken, Pa., 2017, www.astm,org), Treatment chambers are treated with gaseous CD dispensed at various treatment rates. The effects of gaseous CD treatment on VOC concentrations are determined by measurement of the VOC concentrations in response to the gaseous CD treatment.

### Example 2

### Determination of TVOCs Emitted from New Carpet Material

Sections of new carpet material (10-20 square feet) are placed in a sealed chamber (35-225 cubic feet) and incubated for 72 hrs at room temperature. The VOC concentration in air is measured at regular time intervals to determine emitted TVOC concentration over the 72 hr period. The average TVOC emission per unit area of carpet is determined for multiple sections of the same particular new carpet material.

### Example 3

### Treatment of New Carpet Material with CD Solution

Sections of new carpet material are treated with CD by applying a CD solution using a sprayer and applying the solution at a rate of 1 gallon per 250 ft² of carpet or by dipping the entire carpet section into the liquid (both of which facilitate saturation of the carpet section with CD solution) and subsequently allowing the carpet section to air dry for 1 hr. Control sections of the same new carpet material are treated with water to account for differences in VOC off-gassing rates between wet and dry carpet. The CD concentration of the treatment solution is varied within a range of from about 0 to 500 ppm CD with a final pH of between 4 and 5. Treatment and control sections are placed in a sealed chamber following treatment and 1 hour of drying and the TVOC concentration from emissions is measured for 72 hours. The TVOC emissions of CD-treated carpet is compared to control samples to determine the efficacy of treatment of carpet materials with different rates of CD applied as a solution on reduction of TVOC emission. Effects of CD treatment on carpet material integrity and color are also evaluated to determine CD application rates that produce reduction of TVOC emission in the absence of deleterious effects of CD on carpet material physical integrity.

### Example 4

### Treatment of TVOCs Emitted from New Carpet Material with Gaseous CD

Sections of new carpet material are enclosed in sealed chambers as describe above. Gaseous CD is dispensed into treatment chambers at rates of 0.4 mg MAO total space and 1 mg CD/ft3 total space. Control chambers are untreated. The VOC concentration in each chamber is measured at regular time intervals to determine emitted TVOC concentration over the 72 hr period.

### Example 5

### Treatment of Odorant-Infused Carpet Material with CD

Sections of carpet material are spotted with known quantities of a chemical odorant and placed in sealed chambers. Concentrations of volatilized odorant present in the sealed chambers are measured at regular time intervals to determine emission rate and concentration. Liquid CD treatments are performed by spraying CD solutions at various CD concentrations on the odorant-treated sections prior to placement in sealed chambers. Gaseous CD treatments are performed by dispensing gaseous CD at various treatment rates in the sealed chambers. The effects of application of liquid and gaseous CD on odorant emission rates and atmospheric concentrations are determined by measurement and comparison of volatilized odorant in controls and treatments.

### Example 6

### Effects of Long-Term Application of Gaseous CD on TVOC Emissions in Newly Constructed and Newly Renovated Buildings

Total VOC concentrations are measured in rooms of newly constructed and newly renovated buildings over an extended time period of 30 to 180 days following completion of construction or renovation. Rooms of comparable sizes and configurations, including fit and finish, air handling, and use or traffic are identified and denominated as treatment and control rooms. Gaseous CD is dispensed in treated rooms at various rates below a permissible concentration for long-term human exposure, such as about 0.1 ppm. TVOC emissions of non-treated and treated rooms are measured and compared to determine the effects of different gaseous CD treatment levels on measured TVOC levels in treated rooms.

### Example 7

### Treatment of Carpet Material to Reduce Microbial Contamination

Sections of carpet material are inoculated with known quantities of various test microorganisms prepared using conventional methods. Inoculated carpet sections are enclosed in sealed chambers as describe above and treated using gaseous CD and liquid CD solutions sprayed on the carpet sections as described above. Inoculated carpet sections in control chambers are untreated. Following CD treatments, carpet sections are sampled using conventional methods such as microvacuum sampling and samples are analyzed to determine the number of viable microorganisms using conventional methods as disclosed herein, for example cell counts, infectivity titers, cell viability assays using immunofluorescence, reverse transcription-quantitative polymerase chain reaction (RT-qPCR), plaque assays, enzyme-linked immunosorbent assays (ELISAs). The number of viable microorganisms detected before treatment is compared to the number of viable microorganisms detected after CD treatment to determine the effectiveness of CD in reducing the number of viable microorganisms.

### Example 8

### Treatment of Carpet Material to Reduce Microorganism Metabolites

Sections of carpet material are inoculated as described above with various microorganisms known to produce microbial metabolites including microbial VOCs (MVOCs) are placed in chambers and incubated under environmental conditions suitable to induce MVOC production. The sections of carpet material are treated with gaseous and liquid CD as described above. Control sections are untreated. Following treatment, sections of carpet material and chambers are sampled to determine the effect of CD treatment on the presence of microorganism metabolites after CD treatment.

It will be understood that the embodiments of the disclosure described above can be modified in myriad ways other than those specifically discussed without departing from the scope of the disclosure. General variations to these embodiments may include different tooth whitening compositions and method steps of applying the compositions.

Those skilled in the art will readily recognize that even though selected preferred embodiments of the disclosure have been depicted and described, it will be understood that various changes and modifications can be made other than those specifically mentioned above without departing from the spirit and scope of the disclosure, which is defined by the claims that follow.

What is claimed is:

1. A method of treating a space to reduce a concentration of a volatile organic compound (VOC) comprising:
determining a first concentration of a first VOC at a first time; and
applying a chlorine dioxide treatment in the space for a treatment period in response to the first concentration of the first VOC;
wherein the chlorine dioxide treatment is effective to reduce the first concentration of the first VOC to a second concentration at a second time that is lower than the first concentration as compared to a non-treated space.

2. The method of claim 1, wherein the second concentration is one of about 10%, or about 15%, or about 20%, or about 25%, or about 30%, or about 35%, or about 40%, or about 45%, or about 50%, or about 55%, or about 60%, or about 65%, or about 70%, or about 75%, or about 80%, or about 85%, or about 90%, or about 95%, or about 100% lower than the first concentration.

3. The method of claim 1, wherein the method further comprises determining a first concentration of a second VOC at a first time, and wherein the chlorine dioxide treatment is effective to reduce the first concentration of the second VOC to a second concentration at the second time that is lower than the first concentration.

4. The method of claim 1, wherein the space comprises one of a residential building interior space and a commercial building interior space.

5. The method of claim 1, wherein the space comprises a carpeted surface, and wherein the applying a chlorine dioxide step comprises one of applying an aqueous chlorine dioxide solution to the carpeted surface and dispersing a gaseous chlorine dioxide solution in the space.

6. The method of claim 1, wherein the space comprises a floor covering, wherein the first VOC is emitted from the floor covering, and wherein the applying a chlorine dioxide step comprises applying a gaseous chlorine dioxide treatment.

7. The method of one of claims 5 and 6, wherein the method is performed subsequent to installation of the floor covering in the space.

8. The method of claim 6, wherein the first VOC is emitted from one of a floor covering material, an underlayment material, and a floor covering adhesive.

9. The method of claim 8, wherein the first VOC is emitted from one of an underlayment material and a floor covering adhesive, and wherein the one of the underlayment material and the floor covering adhesive is positioned beneath a floor covering material.

10. A method of reducing a volatile organic compound (VOC) emission rate of a material comprising:
determining a first VOC emission rate of a material at a first time;
applying a chlorine dioxide treatment to a material to produce a treated material; and
determining a second VOC emission rate of the treated material at a second time;
wherein the chlorine dioxide treatment produces a reduced VOC emission rate of the treated material wherein the second VOC emission rate of the treated material is lower than the first VOC emission rate of the material.

11. The method of claim 10, wherein the method further comprises:
reserving an untreated portion of the material;
determining an untreated material VOC emission rate of the untreated portion of the material at the second time; and
comparing the second VOC emission rate of the treated material to the untreated material VOC emission rate to determine a VOC emission rate reduction effect of the chlorine dioxide treatment.

12. The method of claim 10, wherein the material comprises a carpeting material.

13. The method of claim 12, wherein the chlorine dioxide treatment is suitable to achieve the reduced VOC emission rate without a substantial effect on one of a material color, a material physical integrity, a material physical performance (stain resistance, wear resistance, color fade resistance), and a material physical attribute (strength, softness, flexibility, resiliency).

14. The method of claim 12, wherein the applying a chlorine dioxide treatment is performed at one of a pre-tufting stage (undyed or dyed yarn), a post-tufting and dying stage, a post-drying and pre-finishing stage, a post-finishing and pre-packaging stage, a packaging stage, and a post-installation stage.

15. The method of claim 12, wherein the first time is less than one of about 4 hrs, about 8 hrs, about 12 hrs, about 16 hrs, about 20 hrs, and about 24 hrs after installation of the carpeting material in a space.

16. The method of claim 15, wherein the second time is less than one of about 24 hrs, about 36 hrs, about 48 hrs, about 60 hrs, about 72 hrs, and about 96 hrs.

17. A method comprising treating a substrate with a chlorine dioxide treatment, wherein, the chlorine dioxide treatment is effective to reduce a TVOC emission rate of the substrate, and wherein the chlorine dioxide treatment does not produce a detrimental impact on the substrate.

18. A method comprising:
treating a substrate with a chlorine dioxide treatment in situ in one of a space for a treatment period;
wherein the space comprises one of a residential space or a commercial space;
wherein the chlorine dioxide treatment is compatible with human occupancy of the space during the treatment period,
wherein the chlorine dioxide treatment is effective to reduce an emitted TVOC quantity in the space as compared to an untreated substrate in an equivalent space, and
wherein the chlorine dioxide treatment does not produce a detrimental impact on the substrate.

19. A method comprising:
treating a soft surface substrate comprising a first microorganism located in the soft surface substrate with a chlorine dioxide treatment for a treatment period;
wherein the first microorganism is viable prior to the chlorine dioxide treatment,
wherein the chlorine dioxide treatment is effective to render the first microorganism non-viable, and
wherein the chlorine dioxide treatment does not produce a detrimental impact on the soft surface substrate.

20. The method of claim 19, wherein the chlorine dioxide treatment comprises application of gaseous chlorine dioxide in one of a residential building interior space and a commercial building interior space enclosing the soft surface substrate.

21. A method for reducing a number of microorganisms in a material comprising:
determining a first number of microorganisms in a material at a first time;
applying a chlorine dioxide treatment to a material to produce a treated material; and
determining a second number of microorganisms in the treated material at a second time;
wherein the chlorine dioxide treatment produces a reduced number of microorganisms in the treated material wherein the second number of microorganisms in the treated material is lower than the first number of microorganisms in the material.

22. The method of claim 21, wherein the number of microorganisms is determined using a quantitative assay.

23. A method for reducing a quantity of a microorganism metabolite in one of a material or a space comprising:
determining a first quantity of a microorganism metabolite in one of a material or a space at a first time;
applying a chlorine dioxide treatment to a material to produce one of a treated material and a treated space; and
determining a second quantity of the microorganism metabolite in one of the treated material and the treated space at a second time;
wherein the chlorine dioxide treatment is effective to reduce the quantity of the microorganism metabolite in the one of the treated material and the treated space and wherein the second quantity of the microorganism metabolite is lower than the first quantity of the microorganism metabolite.

24. The method of claim 23, wherein the microorganism metabolite comprises a microbial VOC.
